(19) United States
(12) Patent Application Publication
(10) Pub. No.: US 2011/0189100 A1
(43) Pub. Date: Aug. 4, 2011

(54) BONE SUBSTITUTE CONTAINING A CONTRAST AGENT, METHOD FOR PREPARING SAME AND USES THEREOF

(76) Inventors: Xavier Bourges, Vay (FR); Serge Baroth, Reze (FR); Guy Daculsi, Vigneux De Bretagne (FR)

(21) Appl. No.: 12/739,129

(22) PCT Filed: Oct. 22, 2008
(86) PCT No.: PCT/EP08/64229

§ 371 Date: Apr. 21, 2010

(30) Foreign Application Priority Data
Oct. 22, 2007 (FR) 0758464

(51) Int. Cl. A61K 49/06; A61K 49/00; A61K 49/04; A61P 43/00; B29C 71/02

(52) U.S. Cl. 424/9.3; 424/9.1; 424/9.4; 264/345

(57) ABSTRACT

The invention relates to a composition for biomaterials, characterised in that it comprises a calcium phosphate, in which the molar ratio Ca/P is 1 to 2, sintered with a medical imaging contrast agent uniformly distributed in the composition mass. The invention also relates to a method for preparing the same and to the medical uses thereof.

The present invention relates to bone filling biomaterial which may be used in surgery, which comprises a contrast agent allowing the surgeon to check by medical imaging the success of the implantation of said biomaterial as well as its resorption and substitution by newly formed bone.

Mixtures of hydroxyapatite and of β tricalcium phosphate (β-TCP) form the mineral basis of many filling materials useful in bone surgery. They allow association of the stability of hydroxyapatite, which forms an effective adhesion support for osteoblasts, and of good resorption of β-TCP, by the release of calcium ions by the latter.

The hydroxyapatite and β-TCP matrices may be effectively used in any type of bone filling, notably in orthopedic, cancerological, traumatological, otorhinolaryngological, maxillofacial, parodontological surgery. This material may also be used for treating fractures with substance loss, and pseudo-arthrosis with or without substance loss. It has proved to be also useful for vertebral arthrodeses (fusion of the rachis) and in addition osteotomies. It may also increase the volume of autografts and increase the effects thereof.

Daculsi et al., (Rev. Chir. Orthop. 1989, 75(2), 65-71) have described a material consisting of hydroxyapatite and of β-TCP, (BCP hereafter) with interesting properties.

BCP has an overall porosity of 70% including ⅔ of macroporosity (300-600 μm) and ⅓ of microporosity (pore <10 μm), the latter allowing diffusion of biological fluids.

Phenomena of crystal dissolution (essentially of TCP) cause the release of ions in the biological fluids. This saturation with ions leads to crystalline precipitation (produced in the presence of the patient's own proteins) consisting of biological apatite crystals identical with bone crystals.

This precipitation in the short term allows improvement in the initial mechanical properties, and forms a new interface with the cells and the tissues, which is substituted for the synthetic surface.

Macropores are used for guiding the cells in depth of the implant (osteoconduction), which may resorb the material and instead form differentiated bone tissue.

The neoformed bone rapidly undergoes bone remodeling. A resorption-apposition cycle occurs like in standard bone. The bone volume gradually increases at the expense of BCP. The fast rehabilitation of BCP, by the significance of its porosity, allows an improvement in its mechanical properties during the bone transformation; the BCP implant will acquire the properties of spongious or cortical bone, depending on its implantation site.

It is important to monitor the bone substitution resorption process, required for a real bone substitute. BCP, by its HA and β-TCP balanced mixture, and its micro- and macro-porous structure, allows these kinetics.

One of the main difficulties encountered by the surgeon using materials based on hydroxyapatite and on tricalcium phosphate, lies in the difficulty of monitoring the success of the implantation of the bone substitute immediately after the operation, as well as its resorption and substitution over time by the tissues of the body.

Radio-opacity of hydroxyapatite and of β tricalcium is actually not sufficiently different from that of bone, and therefore does not allow proper viewing of the biomaterial. It would therefore be desirable to include contrast agents in bone substitutes used in surgery.

The obstacles against which went most attempts in this field are the difficulty of obtaining a homogeneous distribution of the contrast agent inside the phosphocalcium matrix on the one hand, and the toxicity of contrast agents existing today, the leakage of the latter into the body being banned on the other hand.

They should also be used in a sufficient concentration in order to be detected, and nevertheless not harm bone colonization. Further, many contrast agents such as BaSO₄ or ZrO₂, have significant secondary effects and may induce pathological bone resorption.

The inclusion of contrast agents in these resorbable prostheses may also make the latter much too hard and damage the joints in the proximity of which they are implanted.

The authors of the present invention have solved the aforementioned problems by surprisingly obtaining a bone substitute comprising calcium phosphate combined with a contrast agent for medical imaging, which induces good bone regrowth without being cytotoxic.

More specifically, the invention relates to a composition for a biomaterial comprising calcium phosphate in which the molar ratio Ca/P is comprised between 1 and 2, sintered with a contrast agent for medical imaging uniformly distributed in the bulk of the composition.

By <<contrast agent>> in the sense of the present invention, is meant a compound capable of generating on a medical image, artificial contrast on an anatomic or pathological structure naturally not very or not at all contrasted, and therefore difficult to differentiate from the neighboring structures. This contrast agent advantageously is a radio-opaque agent useful in X-ray diffraction imaging, i.e. a compound which is very little or not at all crossed by X-rays. The radio-opaque agent is advantageously inorganic or ionic.

By <<molar ratio Ca/P>> in the sense of the present invention, is meant the ratio between the number of calcium atoms and the number of phosphorus atoms in the chemical formula of calcium phosphate.

With the invention, it is advantageously possible to include inorganic or ionic contrast agents in the calcium phosphate matrix.

Calcium phosphate is advantageously a mixture of β tricalcium phosphate and hydroxyapatite, advantageously in a β tricalcium phosphate/hydroxyapatite weight ratio comprised between 20/80 and 70/30, more advantageously equal to 40/60.

It is possible to observe, in the elementary unit cell of calcium phosphate, partial substitution of calcium with the cation from the contrast agent, and/or partial substitution of the phosphate or of another (for example hydroxyl, fluoride, chloride) anion with the anion of the contrast agent.

The contrast agent proportion in the composition according to the invention is advantageously comprised between 2 and 40% of the total weight, more advantageously between 5 and 35%, more advantageously still equal to about 20%.

The contrast agent may be selected from radio-opaque agents for X-ray diffraction imaging and the contrast agents for nuclear magnetic resonance imaging.

The contrast agent is advantageously selected from barium sulfate, bismuth oxide, lutetium oxide, gadolinium phosphate and mixtures thereof.

According to a particular embodiment of the invention, the composition appears as a granule of irregular shape. According to another advantageous embodiment of the invention, the composition appears as a granule with a spherical shape.

Advantageously, the average diameter of the granules of the composition according to the invention is comprised between 20 and 500 μm, preferably between 40 and 300 μm, more preferably between 80 and 200 μm.

The invention also relates to a composition according to the invention as a bone substitute.

The object of the invention is also an injectable composition comprising a composition according to the invention suspended in a hydrogel.

The hydrogel advantageously comprises an aqueous solution of a cellulose derivative, advantageously hydroxypropylmethyl cellulose.

The invention also relates to a composition according to the invention or to an injectable composition according to the invention as a bone substitute.

The composition according to the invention may advantageously be used as bone filling material in any bone reconstruction or regeneration surgery. The composition according to the invention may also be used in implants and osteosyntheses based on polymers and granules, such as for example the resorbable polymers of the PLA-GLA (polylactic acid-polyglycolic acid) family, or non-resorbable polymers such as PEEK (polyether ether ketone).

The injectable composition according to the invention may advantageously be used as a filling material in any bone tissue, reconstruction or regeneration surgery, for example articular and dental, and notably in minimally invasive surgery.

The invention also relates to a method for preparing a composition according to the invention, characterized in that it comprises the following successive steps:
(a) mixing calcium phosphate in which the molar ratio Ca/P is comprised between 1 and 2, advantageously calcium-deficient apatite, with a contrast agent until a uniform distribution of the contrast agent is obtained in the calcium phosphate;
(b) granulating the resulting mixture;
(c) sintering the granules at a temperature above 600° C., advantageously at a temperature comprised between 700 and 1,300° C.;
(d) optionally sterilizing the sintered granules.

The object of the invention is therefore also a composition capable of being obtained according to the method above.

By <<calcium-deficient apatite>> is meant in the sense of the present invention, a non-stoichiometric apatite. The latter may advantageously fit the formula Caₓ(PO₄)_{y}(X)₁₋₂, wherein X may be selected from OH, F and Cl and wherein x is comprised between 7 and 10 excluded, and y between 3 and 6 excluded. It may also have the formula Ca₉(HPO₄)(PO₄)₅OH.

By <<sintering>> in the sense of the present invention, is meant the action of consolidating by action of heat, a more or less compact granular agglomerate with or without melting of one or more of its constituents.

Different granulation techniques may be used for obtaining these granules comprising a contrast agent.

Calcium phosphate, advantageously a calcium-deficient apatite, may be, is prepared, by reaction between a calcium salt (for example calcium nitrate or calcium acetate) and a phosphate salt (for example ammonium phosphate or sodium phosphate) in which the molar ratio Ca/P is comprised between 1 and 2. It is followed by precipitation in an aqueous basic medium at a temperature from 25 to 80° C., and then by a basic maturation phase which allows an increase in the crystallinity and allows the Ca/P ratio of the final apatite to be monitored and increased.

Another synthesis route for calcium phosphate may apply the reaction between calcium hydroxide with a phosphoric acid solution.

The first shaping operation (step a) consists of obtaining a distribution of the contrast agent in the calcium phosphate. This homogenization may be accomplished in an aqueous medium or in dry form, advantageously in an aqueous medium.

It is particularly advantageous to obtain a homogenous mixture, wherein the contrast agent is uniformly distributed within the phosphocalcium matrix, before any heat treatment.

The powder comprising calcium phosphate and the contrast agent may first of all be milled in the centrifugal mill, and dispersed in order to avoid agglomerates. The homogeneous mixture is then advantageously obtained by the use of a three-dimensional (so-called “turbulent”) mixer for dry forms or with a rotary mixer for wet forms. The obtained powder advantageously has a size of less than 20 μm.

Once the homogenous mixture is obtained, granulation of this product may be carried out (step b), by conventional techniques known to one skilled in the art.

Two main routes may advantageously be utilized for obtaining the granules according to the invention: the wet route or the dry route, according to techniques known to one skilled in the art and abundantly described.

The wet route may essentially consist of using water as a binder with the powder obtained in step (a). Mention may be made as an example of wet granulation techniques suitable for carrying out the invention: mechanical fractionation (the use of a wet granulator, the wet material being forced to cross a sieve); direct drying in a hot air flow (use of a spray dryer, the material being dispersed in a liquid and injected into a more or less hot air flow); the fluidized air bed technique; the granulating plate technique; the granulating drum; dispersion of drops (<<prilling>>).

The dry route may consist of agglomerating very fine particles by a specific mechanical movement. By forming chemical, electrostatic, and/or magnetic bonds, and/or by adding a binder, and/or further because of their shape, the particles will tend to nest together. In this case, the compaction technique (press), followed by the milling fractionation technique, may be used (for example by means of a granulating plate, a granulating drum, without adding any binder).

Once the granules are obtained, sintering at high temperature advantageously above 600° C., more advantageously comprised between 700 and 1,300° C., is carried out, in order to decompose the calcium phosphate comprising the contrast agent into hydroxylapatite and β tricalcium phosphate, comprising the contrast agent. This last step may advantageously allow integration of a radio-opaque element, such as for example barium in the elementary unit cell of calcium phosphate.

Sintering may be carried out according to techniques well-known to one skilled in the art. With it, it is also possible to obtain good mechanical strength and a material of the crystalline type.

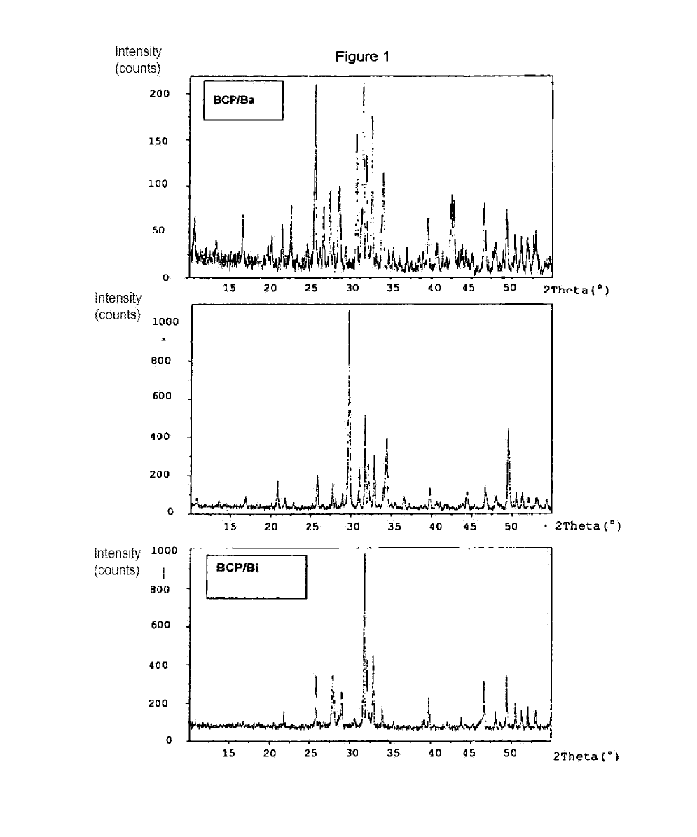

The invention will now be illustrated in a non-limiting way by the example 1-4 and the following FIGS. 1-5.

FIG. 1 illustrates the X-ray diffraction spectra of compositions according to the invention.

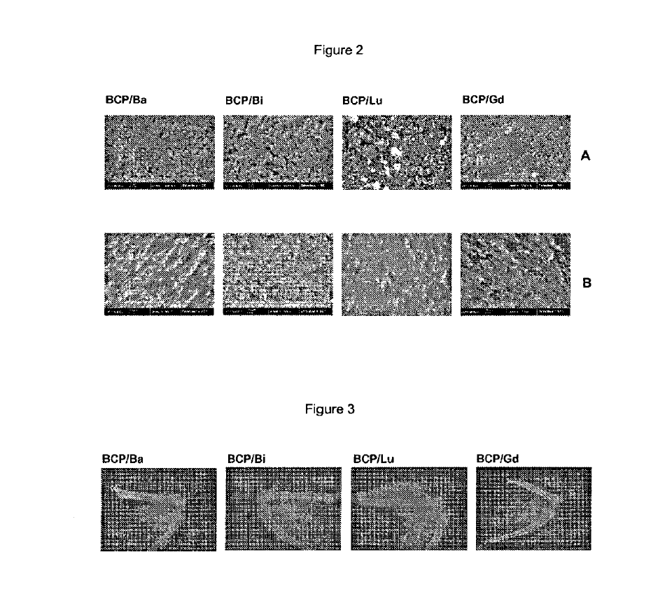

FIG. 2A illustrates the surface of granules according to the invention, as observed by scanning electron microscopy, and FIG. 2B illustrates this same surface after four days of cultivation of MCT3-E1 cells.

FIG. 3 illustrates radiographies of femoral epiphysis of rats in which the radio-opaque granule was implanted, after three weeks of implantation.

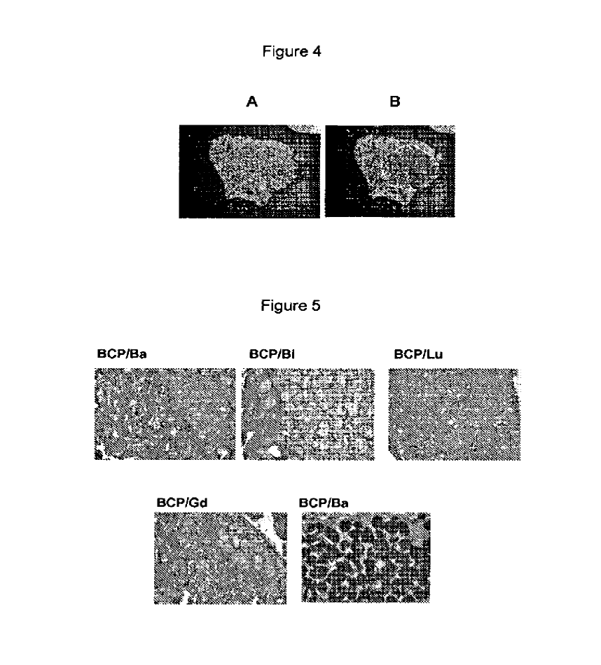

FIG. 4A illustrates an image obtained by scanning electron microscopy of the femoral epiphysis of rats in which a radio-opaque granule was implanted, just after the implantation. FIG. 4B corresponds to processing of the image of this epiphysis after three weeks of implantation in order to view the newly formed bone in white.

FIG. 5 illustrates an image under the polarized light microscope showing the bone regrowth and the tissue colonization in rats after three weeks of implantation.

### EXAMPLE 1

### Preparation of Bone Substitutes Comprising a Contrast Agent

Four radio-opaque compounds were tested: barium sulfate (BaSO₄), lutetium oxide (Lu₂O₃), bismuth oxide (Bi₂O₃) and gadolinium phosphate (GdPO₄). Each of these compounds was mixed in an amount of 20% by weight to calcium-deficient apatite. The composite material was prepared as round granules with an average diameter comprised between 80 and 200 μm. The granules were sintered at 1,050° C., in order to decompose the calcium-deficient apatite into hydroxyapatite and β-TCP in a hydroxyapatite/β-TCP weight ratio of 60/40, and they were then sterilized with steam (121° C., 30 minutes) or under dry conditions (180° C., for four hours) in order to be used in studies in vitro and in vivo.

These granules will subsequently be designated as BCP/Ba, BCP/Lu, BCP/Bi and BCP/Gd.

The diffractograms of BCP/Ba, BCP/Lu, BCP/Bi show modifications of the crystallographic parameters a and c of hydroapatite and of β-TCP, ascribed to the integration of cations and anions of the contrast agent into the unit cell of calcium phosphate. This phenomenon is the most marked in the case of barium.

### EXAMPLE 2

### In Vivo Biocompatibility

The biocompatibility of bone substitutes prepared in the example above was evaluated according to the ISO 10993-5 standard on extracts of MC3T3-E1 osteoblast cells. The materials in this case were prepared, not as granules, but as tablets with a diameter of 10 mm and a thickness of 1 mm by uniaxial compaction. Cytotoxicity was measured by using an MTS test. The results are grouped in the Table 1 below.

TABLE 1
MTS activity of MC3T3-E1 cells after 72 hrs of culture,
according to the ISOP 10993-5 standard.

| | Materials | | | | |
|---|---|---|---|---|---|
| | Plastic | | | | |
| | (control) | Act D | BCP/Ba | BCP/Bi | BCP/Lu |
| Activity of | 100 | 10 | 100 | 98 | 55 |
| the MTS cells | | | | | |
| at 72 hrs (%) | | | | | |

No cytotoxicity was detected for BCP/Ba and BCP/Bi, while BCP/Lu proved to be cytotoxic for osteoblast cells (decrease of MTS activity by more than 20%). Act D is the acronym for actinomycin D at 5 μg/mL (negative control).

### EXAMPLE 3

### Scanning Electron Microscopy

Observation by scanning electron microscopy of the composite surfaces (FIG. 2A) shows that the addition of contrast agents to the calcium phosphate matrix has an influence on the shape of the crystal, on its size and on its microporosity. Thus, addition of BaSO₄ led to the formation of large crystals having low microporosity and a smooth surface.

Addition of Bi₂O₃ led to the growth of needle-shaped crystals having large microporosity in the case of Lu₂O₃ and GdPO₄, the microporosity is large and the crystals are of small size. Many fragments are present on the BCP/Lu₂O₃ matrix.

The morphology of the MC3T3-E1 cells was determined on the fourth day of culture in direct contact with the granules. After fixation on cells, the granules were covered with gold and palladium and observed under the scanning electron microscope with diffraction of back-scattered electrons.

The obtained images are reproduced in FIG. 28.

The type of the matrix has an influence on the development of the cells. The MC3T3-E1 cells have a standard osteoblast morphology in all the cases (long pseudopodia with good spreading), except for the matrix including Bi₂O₃ for which the morphology and the spreading of the cells is found to be changed.

Proliferation of cells on the other hand was found to be considerably affected by the type of granules. Cell proliferation was more significant on BCP/Ba, less significant and approximately similar in the case of BCP/Lu and BCP/Gd and even less on BCP/Bi.

### EXAMPLE 4

### Animal Implantation and Histology

The granules were implanted in the femoral epiphysis of two female rats of the Wistar type. A defect with a diameter of 3 mm and of 5 mm was made and filled with the granules. After three weeks of implantation, radiographies were recorded in order to evaluate radio-opacity.

The obtained images are reproduced in FIG. 3.

Evaluation of the radio-opaque potential in situ of the different materials has shown that BCP/Ba and BCP/Bi have the best contrast relatively to the bone environment.

Next, the implants were taken, fixed in a formol solution and included into glycol methacrylate resin. The blocks were cut into sections with a thickness of 30-100 μm in the microtome with a diamond saw, and analyzed in photon microscopy and in scanning electron microscopy.

The obtained radiographies are reproduced in FIGS. 4 and 5. A quantification of the bone colonization, performed by automated 2D image analysis in scanning electron microscopy (back-scattered electrons, BSE) is given in the table below.

TABLE 2
Quantification of bone colonization in the material

| | | | Surface of |
|---|---|---|---|
| | Total | Biomaterial | neoformed |
| Material | surface (%) | surface (%) | bone (%) |
| BCP/Ba | 100 | 50 | 15 |
| BCP/Bi | 100 | 45 | 7 |
| BCP/Lu | 100 | 44 | 7 |
| BCP/Gd | 100 | 37 | 13 |

The complementary histological results shown in FIGS. 4 and 5 provide an evaluation of the amount of newly formed bone and of the resorption of the granules (FIG. 4), as well as the quality of the bone regrowth in contact with the material (FIG. 5). The BCP/Ba and BCP/Gd granules having the best bone regrowth from a quantitative and qualitative point of view, relatively to BCP/Bi and BCP/Lu.

1. A composition for biomaterials, characterized in that it comprises calcium phosphate in which the molar ratio Ca/P is comprised between 1 and 2, consisting of β tricalcium phosphate and of hydroxyapatite in a β tricalcium phosphate/hydroxyapatite weight ratio comprised between 20/80 and 70/30, sintered with a contrast agent for medical imaging, uniformly distributed in the bulk of the composition.

2. The composition according to claim 1, characterized in that the contrast agent is a radio-opaque compound, preferably an inorganic compound.

3. The composition according to claim 1, characterized in that the β tricalcium phosphate/hydroxyapatite weight ratio is equal to 40/60.

4. The composition according to claim 1, characterized in that the proportion of contrast agent is comprised between 2 and 40% of the total weight, preferably between 5 and 35%, more preferentially further equal to about 20%.

5. The composition according to claim 1, characterized in that the contrast agent is selected from radio-opaque agents for X-ray diffraction imaging and contrast agents for nuclear magnetic resonance imaging.

6. The composition according to claim 1, characterized in that the contrast agent is selected from barium sulfate, barium oxide, lutetium oxide, gadolinium phosphate, and mixtures thereof.

7. The composition according to claim 1, characterized in that it appears as spherical granules.

8. The composition according to claim 1, characterized in that the average diameter of the granules is comprised between 20 and 500 μm, preferably between 80 and 200 μm.

9. An injectable composition comprising a composition according to claim 1, suspended in a hydrogel.

10. The injectable composition according to claim 9, characterized in that the hydrogel comprises an aqueous solution of a derivative of cellulose, preferably hydroxypropylmethyl cellulose.

11. The composition according to claim 1 or 9 as a bone substitute.

12. A method for preparing a composition according to claim 1, characterized in that it comprises the following successive steps:
(a) mixing calcium phosphate in which the molar ratio Ca/P is comprised between 1 and 2, preferably calcium-deficient apatite, with a contrast agent until a uniform distribution of the contrast agent is obtained in the calcium phosphate;
(b) granulating the resulting mixture;
(c) sintering the granules at a temperature above 600° C., preferably at a temperature comprised between 700 and 1,300° C.;
(d) optionally sterilizing the sintered granules.
